(19) United States
(12) Patent Application Publication
(10) Pub. No.: US 2025/0144363 A1
(43) Pub. Date: May 8, 2025

(54) EMOTION-INDUCING DEVICE, EMOTION-INDUCING METHOD, AND DATABASE AND ODOR CLASSIFICATION INDICATORS USED FOR SAID EMOTION-INDUCING DEVICE AND SAID EMOTION-INDUCING METHOD

(71) Applicant: KABUSHIKI KAISHA TOYOTA CHUO KENKYUSHO, Nagakute-shi, Aichi (JP)

(72) Inventors: Kouichirou IWAI, Nagakute-shi (JP); Katsunori KOUDA, Nagakute-shi (JP); Nobuhiko MURAMOTO, Nagakute-shi (JP)

(73) Assignee: KABUSHIKI KAISHA TOYOTA CHUO KENKYUSHO, Nagakute-shi, Aichi (JP)

(21) Appl. No.: 18/837,650

(22) PCT Filed: Dec. 5, 2022
(86) PCT No.: PCT/JP2022/044671

§ 371 Date: Aug. 12, 2024

(30) Foreign Application Priority Data
Apr. 20, 2022 (JP) 2022-069146

(51) Int. Cl. A61M 21/02; A61M 21/00

(52) U.S. Cl. CPC A61M 21/02; A61M 2021/0016; A61M 2205/584

(57) ABSTRACT

An emotion-inducing device includes an inference means that infers a current emotion and an analysis means that obtains, based on a database, odor information bringing the current emotion closer to an expected emotion. The database associates an odor component with an induced emotion that can occur due to the odor component. The emotion-inducing device may further include an emission means that emits at least one type of odor component based on the odor information. The database is configured such that odor components are arranged in association with respective induced emotions. In this case, two or more index values (axes) that can compartmentalize emotions may be used. The emotion-inducing device is realized so that a computer that executes programs is made to cooperate with measurement equipment and odor emission equipment.

### TECHNICAL FIELD

The present invention relates to an emotion-inducing device capable of inducing an expected emotion and also to relevant techniques (excluding those for medical procedures related to the treatment of humans, etc.).

### BACKGROUND ART

People experience various emotions depending not only on their own characteristics (such as personality), but also on their physical conditions and external environments at each point of time, etc. Emotions affect people's judgment, actions, etc., but emotions at each point of time may not necessarily be favorable. For example, emotions such as anger/annoyance can hinder accurate judgment and action etc., and can even be a trigger for danger.

### Prior Art Documents

### Patent Documents

Patent Document 1: JP2019-172615A

### SUMMARY OF INVENTION

### Technical Problem

Patent Document 1 states that emission of α-pinene (an odor component) can increase the arousal level of an operator such as a driver without causing an unpleasant feeling or the like.

Patent Document 1, however, merely discloses the relationship between an odor component and an arousal level, and nothing therein describes the influence of odor components on emotions, how the odor components induce emotions, or the like.

The present invention has been made in view of such circumstances, and an object of the present invention is to provide an emotion-inducing device that can induce people's emotions in a favorable direction and to provide relevant techniques.

### Solution to Problem

As a result of intensive studies to achieve the above object, the present inventors have come up with an idea of inducing human emotions using odors and have then embodied the idea. By developing this achievement, the present inventors have accomplished the present invention, which will be described below.

### Emotion-Inducing Device

(1) The present invention provides an emotion-inducing device including: an inference means that infers a current emotion; and an analysis means that obtains, based on a database, odor information bringing the current emotion closer to an expected emotion. The database associates an odor component with an induced emotion that can occur due to the odor component.

(2) According to the emotion-inducing device (also simply referred to as “the device”) of the present invention, odor information is obtained that can induce a person's emotion at the present moment (current emotion) to a desired emotion (expected emotion). By emitting an odor component based on the odor information, it is possible to bring the person's current emotion, for example, closer to an emotion that is suitable for the situation or environment at the time.

### Emotion-Inducing Method

The present invention may also be understood as an emotion-inducing method (also simply referred to as “the method”). For example, the present invention may provide an emotion-inducing method including: an inference step of inferring a current emotion; an analysis step of obtaining, based on a database, odor information bringing a current emotion closer to an expected emotion, the database associating an odor component with an induced emotion that can occur due to the odor component; and an emission step of emitting at least one type of odor component based on the odor information.

### Database

The present invention may also be understood as a database. For example, the present invention may provide a database configured such that odor components are arranged in association with respective induced emotions that can occur due to the odor components. Compartmentalization of the induced emotions may be performed using at least two or more index values. This database is used in the above-described emotion-inducing device and emotion-inducing method.

### Odor Classification Indicator

The present invention may also be understood as an odor classification indicator. For example, the present invention may provide an odor classification indicator (also simply referred to as “the indicator”) configured such that odor components are arranged in association with respective induced emotions that can occur due to the odor components. The indication may be made on a display connected to a computer or may also be made on paper media, etc. Such an indication may be made on a coordinate plane composed of two index axes that can compartmentalize emotions.

### Others

(1) “Means (unit)” and “step” can be read as interchangeable. For example, a “. . . means” as substitute for a “. . . step” may be a feature of a “product” (such as a device). Likewise, a “. . . step” as substitute for a “. . . means” may be a feature of a “method.”

The “. . . means” and “. . . step” according to the present invention are achieved, for example, by executing a program on a computing machine (computer). The present invention can also be understood as a program required for achieving the present invention, a “data structure” (database) for the program, a storage medium that stores them, etc.

A computer usually includes at least a storage unit and a control unit. The storage unit stores data required for analysis and control. The storage unit is composed, for example, of a volatile memory that temporarily stores data, a non-volatile memory or a hard disk that stores data for a long period of time, and other necessary components. The control unit is composed, for example, of a calculation unit (such as a CPU) that performs calculations based on the data read from the memory unit (e.g., information necessary for inferring emotions, information that associates odor components with induced emotions, etc.), an interface unit that issues commands to an indication unit (display) or external device (e.g., an odor emission unit) connected to the computer (via wired or wireless connection) based on the results of the calculations, and other necessary components.

(2) Unless otherwise stated, a numerical range “x-y” as referred to in the present specification includes the lower limit x and the upper limit y. Any numerical value included in various numerical values or numerical ranges described in the present specification may be selected or extracted as a new lower or upper limit, and any numerical range such as “a-b” can thereby be newly provided using such a new lower or upper limit.

### BRIEF DESCRIPTION OF DRAWINGS

FIG. 1 is a flowchart of an emotion-inducing process achieved by a computer.

FIG. 2A illustrates examples of measurement items used to infer a current emotion.

FIG. 2B illustrates an example of a database used to analyze odor information.

FIG. 3 illustrates an example of the process for creating a database composed of odor components and induced emotions.

FIG. 4 illustrates an example of an odor classification indicator.

### EMBODIMENTS FOR CARRYING OUT THE INVENTION

One or more features freely selected from the present specification can be added to the above-described features of the present invention. The content described in the present specification can be appropriately applied to any of the device, method, indicator, program, database, etc. of the present invention.

### Inference Means/Step

The emotion (current emotion) at the time when starting to induce the emotion (current situation) is inferred. The inference can be made from at least one of biometric information of the person to be targeted (referred to as a “subject”), information on the environment where the subject is present, self- information of the subject (evaluation, analysis, declaration), etc. Specifically, it is as follows.

The biometric information is obtained, for example, by measuring at least one of the brain wave, brain activity, pupil, facial temperature, facial expression, pulse wave, heartbeat, body temperature, breathing, movement, etc. Brain activity is measured, for example, by optical topography (NIRS) or the like. Heartbeat is measured, for example, by an electrocardiogram or the like. Movement is obtained, for example, by measuring the acceleration, angular velocity, angle (tilt), etc.

The environmental information is obtained, for example, by measuring at least one of the temperature, humidity, light illuminance, color temperature, CO₂ concentration, concentration of volatile organic compounds (VOCs), dust amount, etc.

The correlation between biological information and emotions (database) is elucidated, for example, by Ekman. P., 1992a Are there basic emotions? Psychol. Rev. 99. 550-553. The correlation between environmental information and emotions (database) is elucidated, for example, by I. Zhu Y, Yang M, Yao Y, et al., Effects of Illuminance and Correlated Color Temperature on Daytime Cognitive Performance, Subjective Mood, and Alertness in Healthy Adults. Environment and Behavior. 2019; 51 (2): 199-230.

At least one or more types of information acquired from the biological information, environmental information, self-information, etc. are compared with the corresponding database to infer the emotion at that time (current emotion). Starting from this inferred current emotion, the odor information necessary to induce the desired emotion (expected emotion) is calculated, selected, specified, and so on.

### Analysis Means/Step

Odor information that brings the current emotion closer to the expected emotion is obtained. This analysis is performed, for example, based on a database that associates an odor component with an induced emotion that can occur due to the odor component.

### (1) Database

The database is obtained, for example, by repeatedly testing subjects (panelists) who smell odor components to check what emotions (mental states) are induced. The greater the types of odor components, the number of subjects, the number of tests, etc., the higher the reliability of the database.

There are a plurality of types of induced emotions, such as being happy, angry, sad, and relaxed. To facilitate information processing by a computer, etc., each emotion may be compartmentalized, expressed (identified), and so on, using, for example, at least two or more index values (axes). In this case, the odor components may be arranged (coordinated) in correspondence with respective induced emotions compartmentalized by the index values (axes), and a database can thus be obtained.

The index values (axes) represent parameters for compartmentalizing (classifying) emotions. Depending on the number of compartmentalization of emotions, the type, digitalization, etc. of index values can be appropriately selected. For example, in the case of four emotions as described above, two types of index values may be used to compartmentalize them according to their positive/negative (+/−), large/small, good/bad, etc.

Whether to label the index values (axes) is not limited, but for example, referring to Russell's circumplex model (Arousal, Valence) used to express emotions, one index value (axis) may represent an arousal level (Activated) while the other index value (axis) may represent a pleasantness level (Pleasant). In this case, examples of correspondence relationships include (Pleasant, Activated)→Happy (first quadrant), (Unpleasant, Activated)→Angry (second quadrant), (Unpleasant, Deactivated)→Sad (third quadrant), and (Pleasant, Deactivated)→Relaxed (fourth quadrant).

### (2) Analysis

Odor information that can bring the current emotion closer to the expected emotion is obtained based on the database. For descriptive purposes, an example will be described in which four emotions are assigned to four respective domains (quadrants) divided by the above-described two index values (axes).

The quadrant (i.e., emotion) in the database to which the current emotion estimated by the inference means (step) belongs is recognized. An odor components in the adjacent or opposite quadrant to that quadrant is extracted (selected). For example, when the current emotion is in a “Sad” state (fourth quadrant) and the expected emotion is “Happy,” an odor component associated with “Happy” on the opposite side of “Sad” is extracted. When inducing the emotion from the current emotion “Sad” to the expected emotion “Happy,” the emotion may be made to go through “Angry” (second quadrant) or “Relaxed” (fourth quadrant). That is, after an odor component associated with “Angry” or “Relaxed” adjacent to “Sad” is extracted, an odor component associated with “Happy” adjacent thereto may be further extracted.

When a plurality of odor components are associated with one emotion (quadrant), the odor components within the range corresponding to that emotion may be arbitrarily extracted, or the odor components may also be extracted taking into account a preliminarily set extraction order, an index (weight) corresponding to the current emotion, etc.

It is sufficient that the odor information allows at least one or more types of odor components to be specified, but the odor information may also include the emission amount, emission interval, and emission timing of an odor component, the emission order (preparation) of a plurality of odor components, etc. The expected emotion may be, for example, automatically or fixedly determined according to the environment (e.g., driving, working, resting, etc.), or may also be selectively determined each time by the subject.

### Emission Means/Step, Evaluation Means/Step

At least one type of odor component may be emitted based on the odor information. The emission method, emission amount, etc. may be determined taking into account the person's condition, environment, etc.

Furthermore, the emotion after the odor component is emitted may be evaluated. This evaluation may be performed, for example, through waiting for a predetermined time to elapse after the odor component is emitted and inferring the current emotion as described previously. When the closeness to the expected emotion is insufficient, change of the odor component to be emitted, reanalysis of the odor information, etc. may be repeatedly performed.

### Odor Classification Indicator

According to the odor classification indicator, odor components that are effective in inducing the current emotion to the expected emotion can be visually or intuitively perceived. The indication object (medium) is not limited.

When the odor classification indicator is configured such that odor components that can cause contrasting induced emotions are arranged, for example, in symmetrical domains, odor components that are effective in inducing the expected emotion can be more easily perceived. The odor components are easy to see when they are arranged in an annular or circular shape or when they are color-coded for each odor component and/or for each induced emotion. The indicator may also be provided with two index axes that can compartmentalize emotions. In other words, the odor components and/or the induced emotions may be indicated on a coordinate plane composed of such index axes.

### Others

The subjects may be, but are not limited to, drivers or passengers of mobile objects (such as various vehicles, ships, and aircraft), various workers, etc.

The emission of an odor component may, for example, be performed in a range in which the odor intensity based on the six-stage odor intensity indication method is 3 or less in an embodiment or 2 or less in another embodiment. The lower limit is preferably 0 (odorless) or more (or more than 0) in an embodiment or 1 or more in another embodiment. The relationship between the odor intensity and the concentration of an odor component depends on the odor component. For example, for α-pinene or the like, the concentration is 0.01 ppm or less at odor intensity 0, 0.01-0.25 ppm at odor intensity 1, 0.25-4 ppm at odor intensity 2, 4-30 ppm at odor intensity 3, 30-100 ppm at odor intensity 4, and 100 ppm or more at odor intensity 5. As used in the present specification, “ppm” refers to the volume ratio of an odor component per unit volume, and is measured (specified) by the concentration of the air inhaled into the nose.

The emission of an odor component may be continuous or intermittent (intermissive). The odor component is retained, for example, in a container, absorbent, etc., and the emission/no emission of the odor component is switched by an opening/closing operation or the like of a valve controlled by a computer.

### EXAMPLES

The present invention will be described in more detail while illustrating specific examples of a device (method) that uses odors to induce a person's emotions in a desired direction.

### Emotion-Inducing Device

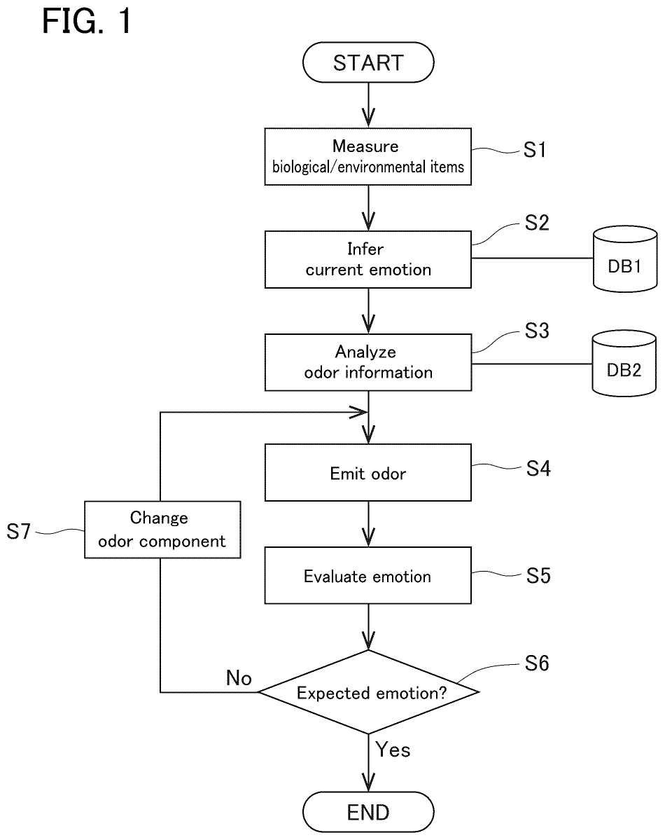

The emotion-inducing device includes a computer, measurement equipment that outputs measured values to the computer, and emission equipment that emits an odor component in response to the input (commands) obtained from the computer. The computer includes a memory unit that stores programs and databases and a control unit that executes programs read from the memory unit. The emotion-inducing device operates, for example, as illustrated in FIG. 1, by the execution of the programs by the computer. Specifically, it is as follows.

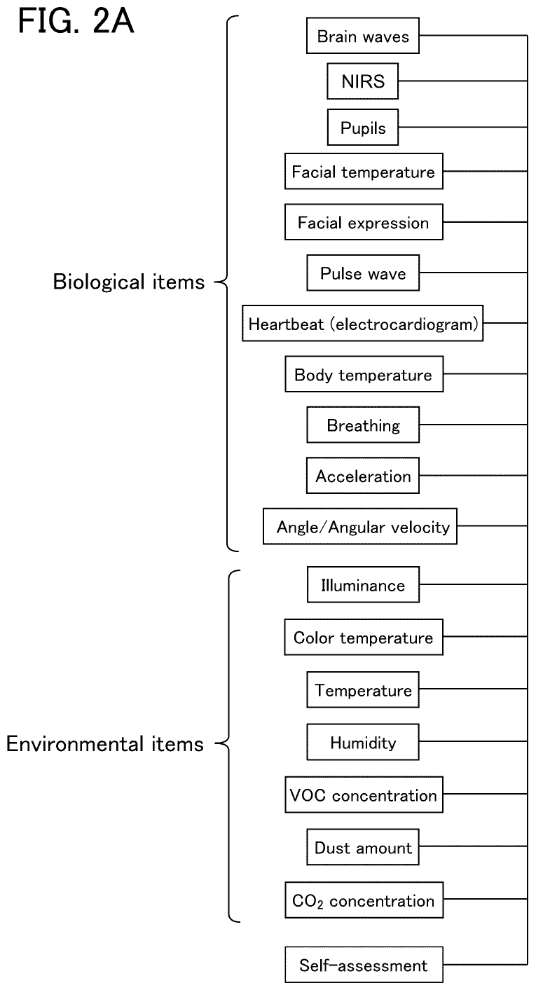

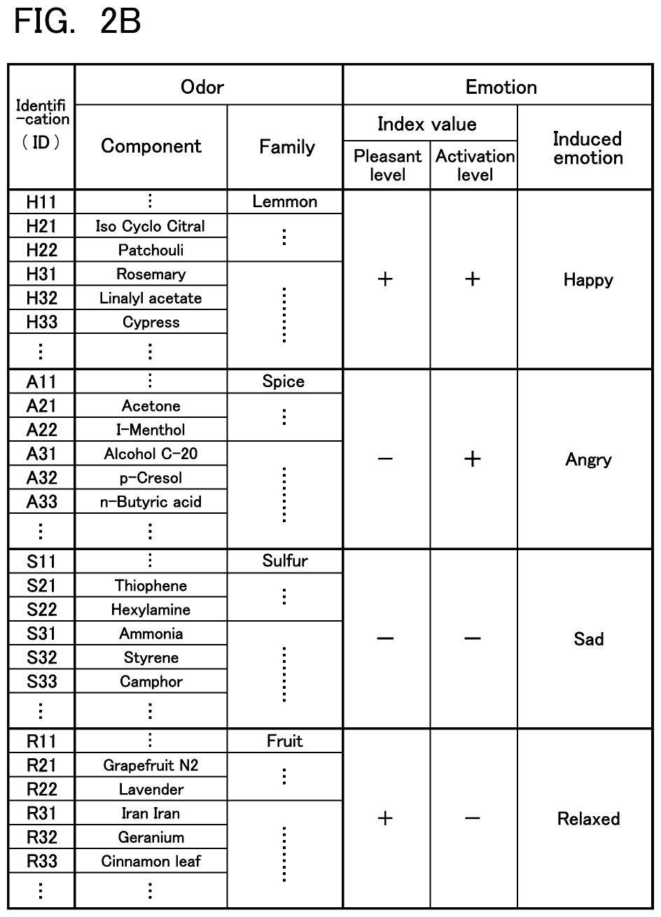

In step S1, the measurement equipment is operated to collect biological information and environmental information of the subject. Examples of the biological information and environmental information are illustrated in FIG. 2A. At the very least, one or more biological information items and representative environmental information items (such as temperature and humidity) may be collected. Additionally or alternatively, the subject may make a self-assessment of his/her emotions and input it to the computer. The more types of information items there are, the easier it is to accurately estimate the subject's current emotion.

In step S2, the computer infers the subject's current mental state (current emotion) based on the collected information obtained in step S1. At this time, the computer predicts the subject's current emotion by applying the collected information obtained by the measurement equipment, etc. to a database DB1 read from the storage unit (comparing the collected information with the database DB1). The database DB1 is a collection of data in which results obtained by repeating various experiments/evaluations are made into a format suitable for the specs, uses, etc. of the emotion-inducing device.

In step S3, the computer calculates odor information for inducing the subject to a mental state (expected emotion) suitable for the current situation based on the current emotion inferred in step S2. Specifically, the computer reads out a database DB2, for example, as illustrated in FIG. 2B, from the storage unit. Then, the computer extracts and selects, from the database DB2, odor components that belong to an induced emotion different from the subject's current emotion.

The induced emotions are classified into the positive (+1) and negative (−1) based on the pleasantness level and the arousal level. Odor components are extracted that belong to an emotional domain in which the sign of at least one of the pleasantness level and arousal level is different with respect to the emotional domain (quadrant) to which the current emotion belongs. At this time, odor components that belong to two or more emotional domains may be extracted, or two or more odor components that belong to one emotional domain may be extracted. Extraction of odor components may be performed in accordance with individual IDs of the odor components or with the odor family (the ID's central character) to which the odor components belong. At this time, a group of two or more types of extracted odor components (candidate group) may be formed. In FIG. 2B, for the sake of simplicity, the pleasantness level and arousal level are set to positive (+1) or negative (−1) so that only the emotional domain or domains to which the odor components belong is specified. However, the pleasantness level and arousal level of each odor component may be expressed as specific numerical values (including positive and negative signs), and the emotional domain to which the odor component belongs and its contribution to the induced emotion (induction degree) may be specifically indicated for each odor component.

The expected emotion may be predetermined based on the environment or the like in which the emotion-inducing device is installed, or may be determined by the computer based on the environmental information or the like related to the subject, or may be set by the subject himself/herself. The odor information may include the type of an odor component as well as its emission amount, emission interval, emission timing, etc.

In step S4, the computer instructs the emission equipment to emit the odor component based on the odor information obtained in step S3. The odor component to be emitted in this step may be of a single type or a combination of two or more types.

In step S5, the computer evaluates the emotion of the subject after the odor component is emitted. This emotion evaluation is performed in the same manner as in the inference of the current emotion described in step S2.

In step S6, the computer compares the emotion evaluated in step S5 with the expected emotion. When the emotion of the subject is close to the predetermined expected emotion, the emission of the odor component is terminated.

On the other hand, when the emotion of the subject is not close to the predetermined expected emotion or its extent is insufficient, the process proceeds to step S7, in which the odor component to be emitted is changed, and step S4 and subsequent steps are repeated. The change of the odor component may be performed, for example, sequentially along the IDs of the odor components from the candidate group obtained in step S3. Alternatively, an odor component that can cause an induced emotion symmetric to the current emotion may be once emitted to bring the emotion of the subject to a neutral state, and then an odor component that can cause the desired expected emotion may be newly emitted. The upper limit of the number of times the odor component is changed (the number of repetitions) may be preliminarily restricted, or may be limited to the change of odor components within the above-described candidate group. Additionally or alternatively, the degree of closeness of the current emotion to the expected emotion may be calculated, and the change of the odor component, adjustment of the emission amount, etc. may be performed based on the degree of closeness.

### Database/Odor Classification Indicator

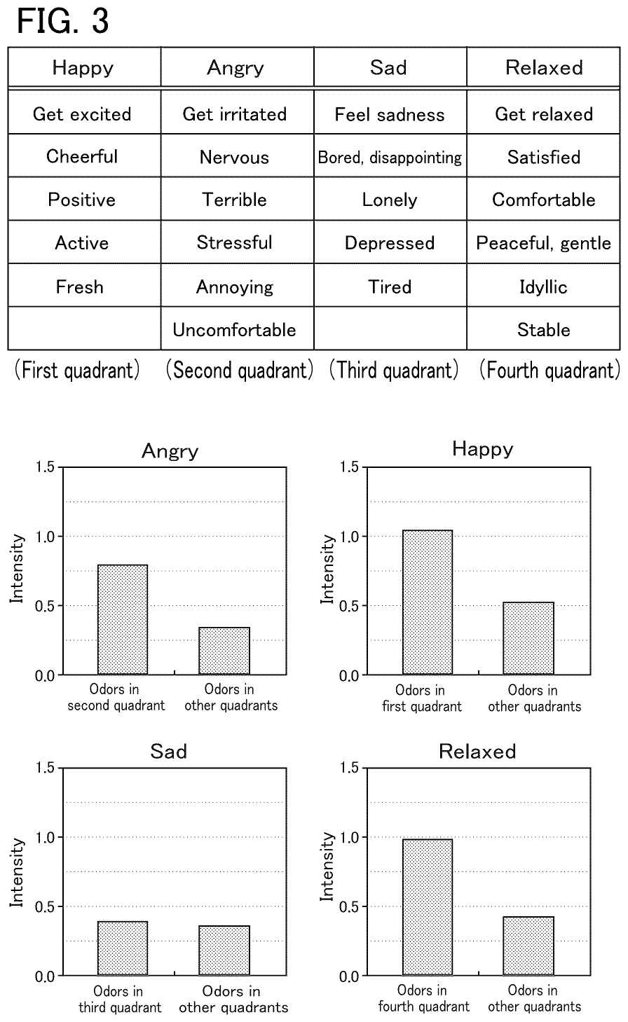

A database that represents the correlation between odor components and induced emotions (see FIG. 2B, etc.) is obtained such that, for example, as illustrated in FIG. 3, a large number of panelists evaluate emotions that occur due to each odor component. Specifically, the database is completed by allocating each odor component to the emotional domain (quadrant) that is most strongly felt by the odor component.

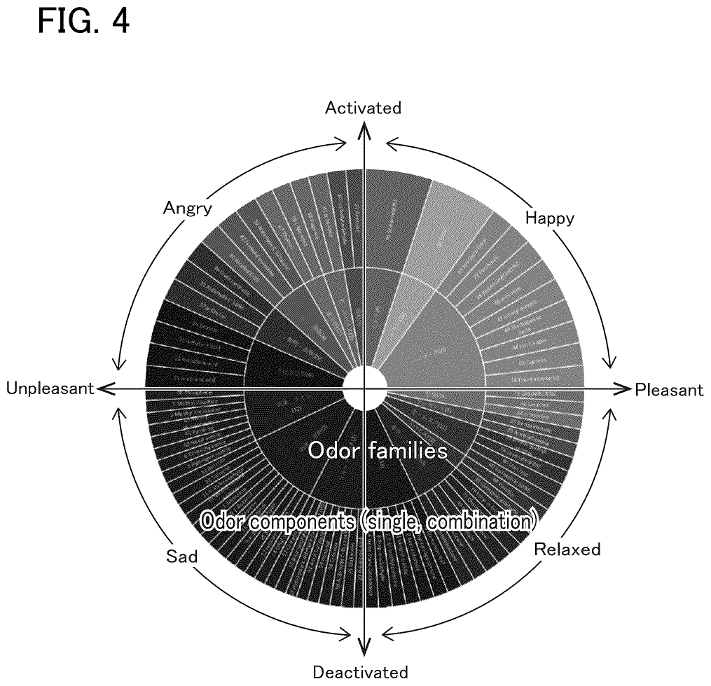

The correlation between odor components and induced emotions may also be expressed as an odor classification indicator illustrated in FIG. 4. This allows the odor components necessary for emotion induction to be intuitively perceived. The odor classification indicator may be expressed by arranging the odor components in an annular or circular shape on an orthogonal coordinate plane composed of an index axis representing the arousal level and an index axis representing the pleasantness level. In this case, the induced emotions are assigned to respective quadrants of the coordinate plane. Furthermore, the odor components are assigned to a quadrant (emotional domain) to which the corresponding induced emotions belong. Individual odor components are indicated on the outer periphery. The family to which each odor component belongs may also be indicated on the inner periphery. In particular, contrasting emotions may be indicated in quadrants (emotional domains) symmetrical to the coordinate center, and odor components may be indicated symmetrically corresponding to respective emotions.

1. An emotion-inducing device comprising:
an inference means that infers a current emotion; and
an analysis means that obtains, based on a database, odor information bringing the current emotion closer to an expected emotion, the database associating an odor component with an induced emotion that can occur due to the odor component.

2. The emotion-inducing device according to claim 1, further comprising
an emission means that emits at least one type of odor component based on the odor information.

3. The emotion-inducing device according to claim 1, wherein the database is configured such that odor components are arranged in association with respective induced emotions using at least two or more index values that can compartmentalize emotions.

4. An emotion-inducing method comprising:
an inference step of inferring a current emotion;
an analysis step of obtaining, based on a database, odor information bringing the current emotion closer to an expected emotion, the database associating an odor component with an induced emotion that can occur due to the odor component; and
an emission step of emitting at least one type of odor component based on the odor information.

5. The emotion-inducing method according to claim 4, further comprising
an evaluation step of evaluating the current emotion after the emission step.

6. A database for use in the emotion-inducing method according to claim 4, wherein the database is configured such that odor components are arranged in association with respective induced emotions that can occur due to the odor components, wherein the odor components are arranged using at least two or more index values that can compartmentalize emotions.

7. The database according to claim 6, wherein the index values include an arousal level and a pleasantness level.

8. An odor classification indicator configured such that odor components are arranged in association with respective induced emotions that can occur due to the odor components.

9. The odor classification indicator according to claim 8, wherein the odor components that can cause contrasting induced emotions are arranged in symmetrical domains.

10. The odor classification indicator according to claim 8, wherein the odor classification indicator is color-coded for each of the odor components and/or each of the induced emotions.

11. The odor classification indicator according to claim 8, wherein the odor components are arranged in an annular or circular shape.

12. The odor classification indicator according to claim 8, wherein the odor components are indicated on a coordinate plane composed of two index axes that can compartmentalize emotions.
